(19) United States
(12) Patent Application Publication
(10) Pub. No.: US 2021/0178076 A1
(43) Pub. Date: Jun. 17, 2021

(54) DRUG DELIVERY SYSTEM WITH DRUG DIFFERENTIATION FEATURE

(71) Applicant: Novo Nordisk A/S, Bagsvaerd (DK)

(72) Inventor: Nicolai Michael Jensen, Koebenhaven SV (DK)

(73) Assignee: Novo Nordisk A/S, Bagsvaerd (DK)

(21) Appl. No.: 17/269,319

(22) PCT Filed: Aug. 26, 2019
(86) PCT No.: PCT/EP2019/072719

§ 371 Date: Feb. 18, 2021

(30) Foreign Application Priority Data
Aug. 27, 2018 (EP) 18190926.8
Oct. 10, 2018 (EP) 18199595.2

(51) Int. Cl. A61M 5/24; A61M 5/315

(52) U.S. Cl. CPC A61M 5/2448; A61M 2205/276; A61M 2005/2488; A61M 5/31511

(57) ABSTRACT

A cartridge comprising a generally cylindrical cartridge main body with a proximal edge portion having a circumferential inner surface and a proximal opening surrounded by a circumferential edge, a piston slidable received therein, and a ring-formed code member attached to the circumferential edge portion and comprising an axially extending mounting portion in engagement with the edge portion circumferential inner surface, a flange portion having a distally facing mounting surface in engagement with the circumferential edge and a proximally facing stop surface, and a drive opening allowing a piston rod to engage the piston proximal surface, wherein the flange portion radially extends within the circumferential boundaries of the cartridge main body.

The present invention generally relates to a medical drug delivery system. In specific embodiments the invention relates to a drug delivery system adapted to ensure application of a correct drug when operated by the user. The present invention also relates to a cartridge for such a system.

### BACKGROUND OF THE INVENTION

In the disclosure of the present invention reference is mostly made to the treatment of diabetes by delivery of insulin-containing drugs, however, this is only an exemplary use of the present invention.

Drug delivery devices (typically for subcutaneous injection) have greatly improved the lives of patients who must self-administer drugs and biological agents. Drug injection devices may take many forms, including simple disposable devices that are little more than an ampoule with an injection means or they may be highly sophisticated electronically controlled instruments with numerous functions. Regardless of their form, they have proven to be great aids in assisting patients to self-administer injectable drugs and biological agents. They also greatly assist care givers in administering injectable medicines to those incapable of performing self-injections.

In particular pen-style injection devices have proven to provide an accurate, convenient, and often discrete, way to administer drugs and biological agents, such as insulin. Modern devices have become more sophisticated, and often include diverse and robust functions, such as memories for remembering time and amount of last dose, as well as, in the case of insulin devices, blood glucose monitors. While pen-style injection devices are typically cylindrically shaped with a needle protruding from the most distal portion of one end of the device, some devices have other shapes with the needle no longer protruding from the most distal part of an end of the device, e.g. Innovo® and InnoLet® from Novo Nordisk A/S, Bagsvaerd, Denmark.

Typically injection devices use a pre-filled cartridge containing the medication of interest, e.g. 1.5 or 3.0 ml of insulin or growth hormone. The cartridge is typically in the form of a generally cylindrical transparent ampoule with a needle pierceable septum at one end, and an opposed piston designed to be moved by the dosing mechanism of the injection device. The injection devices generally are of two types: “Durable” devices and “disposable” devices. A durable device is designed to allow a user to replace one cartridge with another cartridge, typically a new cartridge in place of an empty cartridge. In contrast, a disposable device is provided with an imbedded cartridge which cannot be replaced by the user without damaging or destroying the device, when the cartridge is empty the entire device is intended to be discarded. Most injection devices are provided with a releasable pen cap covering the cartridge and the needle mount portion (see below), this allowing the user to inspect the content of the cartridge by removing the cap.

Often injection devices are provided as a system or family of devices containing different types of drugs, e.g. as known from WO 2004/069314. This application discloses a system of substantially identical injection devices, each individual injection device comprising a housing accommodating an ampoule containing drug sufficient for a number of injections and a dose setting mechanism by which a predetermined dose size can be set, and wherein each of the plurality of injection devices has a different predetermined dose size. The difference in the predetermined dose sizes can in one embodiment be based on the drug in the devices having different strength.

People suffering from diabetes are often treated with multiple daily injections in a regimen comprising one or two daily injections of long acting insulin to cover the basal requirement supplemented by bolus injections of short or rapid acting insulin to cover requirements related to meals.

A user will therefore often require two different injection devices, one containing the long acting insulin and another containing the short or rapid acting insulin. Often these injection devices have different colour indications to inform the user of the kind of insulin contained in the injection device. For example, the FlexPen® system offered by Novo Nordisk comprises pens for long and short acting insulins as well as for mixed insulin, the bodies and caps being identical with colour markings on the main body to differentiate the two types of insulin. In the SoloStar® system offered by Sanofi-Aventis the pens for long and short acting insulins have differently coloured bodies as well as caps. In addition these injection devices can be provided with tactile means such as a mechanical coding informing the user of the kind of insulin contained in the injection device.

US 2010/0042054 A1 discloses a medical delivery system including a pen type injector with a housing consisting of two parts where a proximal part contains a dosing and injection release mechanism and where the distal end houses a cartridge. The cartridge in the distal end is irreplaceably connected to the distal housing. Corresponding mechanical coding features are provided between the distal and proximal housing parts to ensure that only a correct assembly of housing and cartridge can be connected to the proximal part, to ensure correct dosing of a medical drug. It is a problem with this system that the cartridge itself cannot be interchanged from the distal housing part without losing the security provided by the mechanical coding features.

Although the prior art discloses a number of solutions of how to differentiate similar or otherwise identical drug delivery devices containing different kinds of drugs, there is still a need for drug delivery devices and systems which in a simple, reliable and cost-effective manner provides a strong identification of the kind of drug contained in a specific device, this reducing the risk of a user inadvertently taking the incorrect kind of drug.

Having regard to the above, it is an object of the present invention to provide a drug delivery device, system and components therefore which in a safe, simple and cost-effective manner reduces the likelihood of a user taking the incorrect kind of drug.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a first aspect of the invention a cartridge is provided comprising a cartridge body comprising a generally cylindrical main portion defining a longitudinal axis, a distal outlet portion with an outlet opening, and a proximal edge portion having a circumferential inner surface and a proximal opening surrounded by a circumferential edge. A needle penetratable septum is arranged to close and seal the outlet opening. A piston a slidable received in the cylindrical main portion and comprises a proximal free surface, a variable-volume reservoir being formed between the septum and the piston member. The cartridge further comprises a ring-formed code member attached to the circumferential edge portion, the code member comprising an axially extending mounting portion in engagement with the edge portion circumferential inner surface, a flange portion having a proximally facing stop surface, and a drive opening allowing a piston rod to engage the piston proximal surface, wherein the flange portion radially extends within the circumferential boundaries of the cartridge main portion. The flange portion may have a distally facing mounting surface in engagement with the circumferential edge.

By this arrangement an essentially unmodified standard cartridge (merely provided with an additional code member) can be received in an unmodified cartridge holder, this allowing a coded cartridge to be provided in a simple and cost-effective manner.

Whereas a code member may be manufactured from a polymeric material with narrow tolerances the cartridge body is in most cases manufactured from glass and thus comes with much larger tolerances. In the present context this means that although the code member may be dimensioned to have a flange portion which radially extends within the circumferential boundaries of the cartridge main portion a small fraction of the cartridges will statistically have a diameter being actually smaller than the code member.

In an exemplary embodiment the flange stop surface has a non-planar configuration comprising a code pattern of axially extending code structures.

In a further aspect of the invention a drug delivery system is provided, comprising a first cartridge, a second cartridge, and a first drug delivery device. The first and second cartridges correspond to the above-described cartridge, the first cartridge comprising a code member having a first drive opening with a first code configuration, and the second cartridge comprising a code member having a second drive opening with a second code configuration. The first drug delivery device comprises a cartridge holder adapted to receive either of the first and the second cartridge, a first piston rod having a distal end adapted to engage and axially displace a piston in a loaded cartridge in a distal direction to thereby expel a dose of drug from the cartridge, the first piston rod distal end having a first code configuration, and a drive assembly adapted to move the first piston rod in the distal direction, wherein the first piston rod code configuration allows the first piston rod to be inserted through the first drive opening but prevents it from being inserted through the second drive opening.

In an exemplary embodiment the drug delivery system further comprises a second drug delivery device, comprising a cartridge holder adapted to receive either of the first and the second cartridge, a second piston rod having a distal end adapted to engage and axially displace a piston in a loaded cartridge in a distal direction to thereby expel a dose of drug from the cartridge, the second piston rod distal end having a second code configuration, and a drive assembly adapted to move the second piston rod in the distal direction. The first piston rod code configuration allows the first piston rod to be inserted through the first drive opening but prevents it from being inserted through the second drive opening, and the second piston rod code configuration allows the second piston rod to be inserted through the second drive opening but prevents it from being inserted through the first drive opening.

In a yet further aspect of the invention a drug delivery system is provided, comprising a first cartridge, a second cartridge, and a first drug delivery device. The first and second cartridges correspond to the above-described cartridge, the first cartridge comprising a code member having a first stop surface with a first code configuration, and the second cartridge comprising a code member having a second stop surface with a second code configuration. The first drug delivery device comprises a housing, a cartridge holder adapted to receive either of the first and the second cartridge, the cartridge holder being actuatable between (i) a receiving state in which a cartridge can be inserted and received in an axial direction, and (ii) a holding state in which an inserted cartridge is held in an operational position. The first drug delivery device further comprises a piston rod having a distal end adapted to engage and axially displace a piston in a loaded cartridge in a distal direction to thereby expel a dose of drug from the cartridge, a drive assembly arranged in the housing and adapted to move the first piston rod in the distal direction, and a first distally facing mating structure having a first mating code configuration adapted to axially engage a cartridge stop surface code configuration. The first mating code configuration allows the first stop surface to be received in the first mating structure to an axial position allowing the cartridge holder to be actuated from the receiving state to the holding state, but prevents the second stop surface to be received in the first mating structure to an axial position allowing the cartridge holder to be actuated from the receiving state to the holding state.

In an exemplary embodiment the drug delivery system further comprises a second drug delivery device, comprising a housing, a cartridge holder adapted to receive either of the first and second cartridge, the cartridge holder being actuatable between (i) a receiving state in which a cartridge can be inserted and received in an axial direction, and (ii) a holding state in which an inserted cartridge is held in an operational position. The second drug delivery device further comprises a piston rod having a distal end adapted to engage and axially displace a piston in a loaded cartridge in a distal direction to thereby expel a dose of drug from the cartridge, a drive assembly arranged in the housing and adapted to move the first piston rod in the distal direction, and a second distally facing mating structure having a second mating code configuration adapted to axially engage a cartridge stop surface code configuration. The first mating code configuration allows the first stop surface to be received in the first mating structure to an axial position allowing the cartridge holder to be actuated from the receiving state to the holding state, but prevents the second stop surface to be received in the first mating structure to an axial position allowing the cartridge holder to be actuated from the receiving state to the holding state, and the second mating code configuration allows the second stop surface to be received in the second mating structure to an axial position allowing the cartridge holder to be actuated from the receiving state to the holding state, but prevents the first stop surface to be received in the second mating structure to an axial position allowing the cartridge holder to be actuated from the receiving state to the holding state.

In an exemplary embodiment, for each drug delivery device the cartridge holder and the housing comprise corresponding coupling means allowing the cartridge holder to be removed from the housing corresponding to the receiving state and mounted on the housing corresponding to the holding state. The coupling means may be in the form of a bayonet coupling or a threaded coupling.

In a further exemplary embodiment, for each drug delivery device the cartridge holder is non-removable from the housing, the cartridge holder comprising a distal opening allowing a cartridge to be received in a proximal direction, the cartridge holder comprises holding means actuatable from (i) a receiving state allowing a cartridge to be inserted into the cartridge holder, to (ii) a holding state in which a received cartridge is held in an operational position.

The holding means may comprise one or more locking arms each having a distal gripping portion being moved proximally when the holding means is actuated from the receiving state to the holding state.

In a further aspect, objects of the invention are obtained by a cartridge comprising an elongate cartridge body comprising a distal end and a proximal end with a proximal opening surrounded by a circumferential rim portion, needle penetrable septum arranged to close and seal an outlet opening at the distal end, a piston slidable received in the elongate cartridge body and comprising a proximally facing free surface, a variable-volume reservoir being formed between the needle penetrable septum and the piston, and a ring shaped coding plug attached to the proximal circumferential rim portion of the cartridge and comprising a piston rod opening allowing a piston rod to engage the proximally facing free surface of the piston, and mechanical coding structure.

In one embodiment the coding structure of the coding plug is defined by the shape of the piston rod opening having a configuration allowing a correspondingly formed piston rod or piston washer to be inserted there-through.

In a further embodiment the coding structure of coding plug may alternatively or additionally be defined by the dimension (size) of the piston rod opening having a configuration allowing a correspondingly formed piston rod or piston washer to be inserted there-through.

In another embodiment the coding structure of the coding plug is defined by a coding contour formed on a proximally facing circumferential surface of the coding plug surrounding the piston rod opening, and having a configuration allowing mating with a correspondingly formed coding adapter ring surrounding a piston rod of a main drug delivery device.

In a further embodiment, the elongate cartridge body is made of glass and the coding plug is made of a polymeric material.

In a yet further aspect, objects of the invention are obtained by a drug delivery assembly comprising a cartridge according to the first aspect of the invention in combination with a main drug delivery device, the main drug delivery device comprising cartridge holder for axially receiving a cartridge, distal coupling means for attaching a needle assembly, and a proximal body portion formed in extension of the cartridge holder, and including a drug expelling mechanism with a piston rod, wherein a distally facing part of the proximal body portion is configured for mating with the mechanical coding structure on the coding plug, when the cartridge is inserted into the cartridge holder.

In one embodiment, the coding structure on the coding plug of the cartridge is defined by the shape of the piston rod opening having a configuration allowing the correspondingly formed piston rod or piston washer to be inserted there-through.

In a further embodiment the coding structure on the coding plug of the cartridge may alternatively or additionally be defined by the dimension (size) of the piston rod opening having a configuration allowing a correspondingly formed piston rod or piston washer to be inserted there through.

In an alternative embodiment, the coding structure on the coding plug of the cartridge is defined by a coding contour formed on a proximally facing circumferential surface of the coding plug surrounding the piston rod opening, and having a configuration allowing mating with a correspondingly formed coding contour formed on a distally facing circumferential surface of a coding adapter ring surrounding the piston rod of the proximal body portion.

In a yet further aspect, objects of the invention may be obtained by a drug delivery system comprising a first cartridge as defined in connection with the first aspect of the invention, and comprising a first mechanical coding structure, a second cartridge as defined in in connection with the first aspect of the invention, and comprising a second mechanical coding structure, and a main drug delivery device comprising a cartridge holder adapted to axially receive either of the first or the second cartridge, a proximal body portion formed in extension of the cartridge holder, and including a drug expelling mechanism with a piston rod, a third mechanical coding structure formed on the proximal body portion, wherein the first and second cartridge comprise elongate cartridge bodies having identical form and size, and wherein the third mechanical coding structure on the drug expelling mechanism is configured to cooperate with the first mechanical coding structure and to be incompatible the second mechanical coding structure.

Preferably, the first mechanical coding structure and the second mechanical coding structure each is provided on a coding plug attached to a proximal end of the cartridges.

In an embodiment the piston rod has a distal end adapted to engage a piston proximal surface, the distal end of the piston rod comprising the third mechanical coding structure, wherein the third mechanical coding structure: allows distal end of the piston rod to engage the proximal surface of the piston of the first cartridge and prevents distal end of the piston rod from engaging the proximal surface of the piston of the second cartridge.

In an alternative embodiment the third mechanical coding structure is formed a distally facing surface of a coding adapter ring surrounding the piston rod of the proximal body portion, and wherein the third mechanical coding structure: allows complete insertion of the first cartridge into the cartridge holder and prevents complete insertion of the second cartridge into the cartridge holder.

In a further embodiment the system may comprise a second main drug delivery device comprising a cartridge holder adapted to axially receive either of the first or the second cartridge, distal coupling means for attaching a needle assembly, a proximal body portion formed in extension of the cartridge holder, and including a drug expelling mechanism with a piston rod, a fourth mechanical coding structure formed on the proximal body portion, wherein the fourth mechanical coding structure on the drug expelling mechanism is configured to cooperate with the second mechanical coding structure and to be incompatible the first mechanical coding structure.

It should be emphasized that the term “comprises/comprising/comprised of” when used in this specification is taken to specify the presence of stated features, integers, steps or components but does not preclude the presence or addition of one or more other features, integers, steps, components or groups thereof.

As used herein, the term “insulin” is meant to encompass any drug-containing flowable medicine capable of being passed through a delivery means such as a cannula or hollow needle in a controlled manner, such as a liquid, solution, gel or fine suspension, and which has a blood glucose controlling effect, e.g. human insulin and analogues thereof as well as non-insulins such as GLP-1 and analogues thereof. In the description of exemplary embodiments reference will be made to the use of insulin, however, the described module could also be used to create logs for other types of drug, e.g. growth hormone or drugs for haemophilia treatment.

### BRIEF DESCRIPTION OF THE DRAWINGS

In the following embodiments of the invention will be described with reference to the drawings, wherein

FIG. 1 shows a pen device with a housing consisting of two parts where a proximal part contains a dosing and injection release mechanism, where the distal end houses a cartridge, and where the distal end is covered by a pen cap,

FIG. 2 shows the pen device of FIG. 2 with the pen cap removed,

FIG. 3 shows a pen cap and a cartridge holder for a pen device as shown in FIG. 1,

FIG. 4, in a longitudinal cross-section shows a cap mounted on a cartridge holder, and ac coded cartridge mounted in the cartridge holder,

FIG. 5A shows in a side view a coded cartridge with an end plug according to an embodiment of the invention,

FIG. 5B shows in a sectional side view details of a proximal end of the coded cartridge of FIG. 5A,

FIG. 6A shows in a sectional side view a coded cartridge with a first type of coding for an injection system according to a first embodiment of the invention,

FIG. 6B shows in a sectional side view a coded cartridge with a second type of coding for an injection system according to the first embodiment of the invention,

FIG. 7A shows in a perspective view a coded cartridge with a variation over the fourth type of coding for an injection system according to an embodiment of the invention

FIG. 7B shows a side view of the coded cartridge shown in FIG. 7A,

FIG. 7C shows a section through the coded cartridge shown in FIG. 7A, and

FIG. 8 shows in a perspective view an embodiment of a front-loaded drug delivery device.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member or element is used for a given component it generally indicates that in the described embodiment the component is a unitary component, however, the same member or element may alternatively comprise a number of sub-components just as two or more of the described components could be provided as unitary components, e.g. manufactured as a single injection moulded part. The term “assembly” does not imply that the described components necessarily can be assembled to provide a unitary or functional assembly during a given assembly procedure but is merely used to describe components grouped together as being functionally more closely related.

Before turning to embodiments of the present invention per se, an example of a drug delivery device/pen assembly will be described, such a device providing the basis for the exemplary embodiments of the present invention.

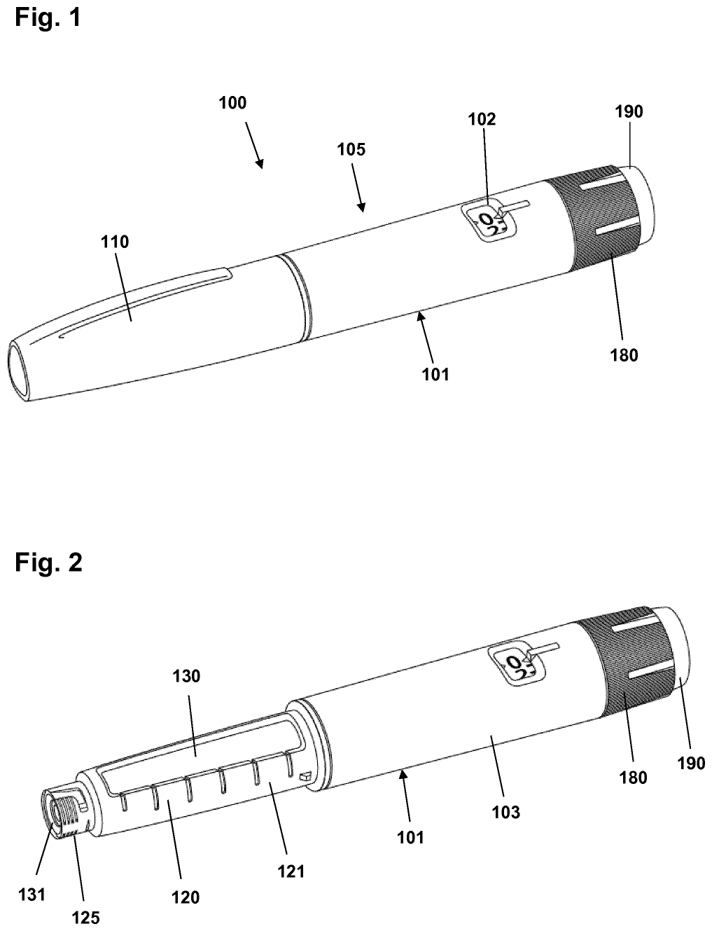

Although the pen-formed drug delivery assembly 100 shown in FIGS. 1-4 represents a “generic” drug delivery device which may be either prefilled or durable, the actually shown device is a FlexTouch® prefilled drug delivery pen as manufactured and sold by Novo Nordisk A/S, Bagsvrd, Denmark. A more detailed description of such a device can be found in e.g. WO 2014/161952, which is hereby incorporated by reference.

The pen assembly 100, shown in FIG. 1, comprises a cap part 110 and a main drug delivery device (or part) 105 having a proximal body portion 101 (which may also be termed a drive assembly portion) with a housing 103 in which a drug expelling mechanism is arranged or integrated. The pen assembly further comprises a distal cartridge holder 120, see FIG. 2, in which a drug-filled transparent cartridge 130 with a distal drug outlet in the form of a needle-penetrable septum 131 is arranged and retained in place by the non-removable cartridge holder 120.

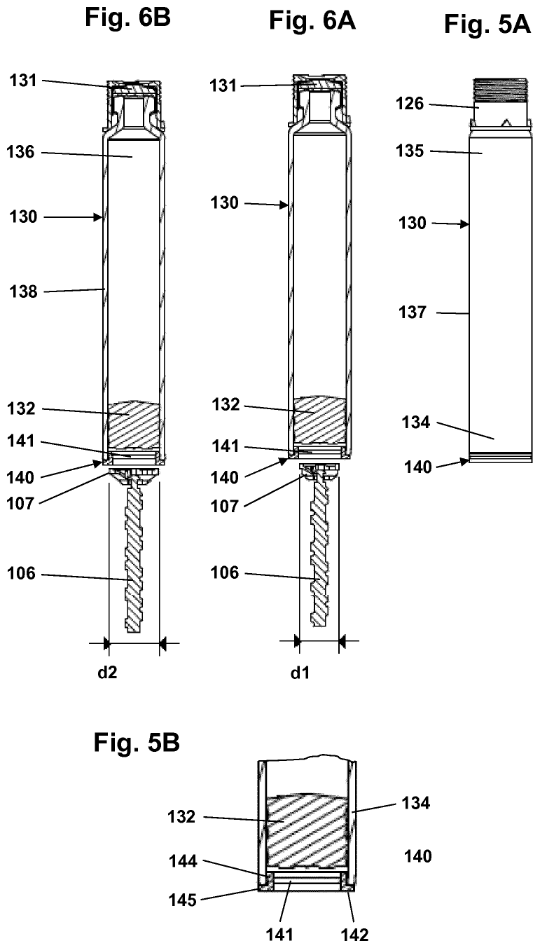

The distal cartridge holder 120 is attached to the proximal body portion 101 and has in the shown embodiment openings allowing a portion of the cartridge 130 to be inspected as well as distal coupling means 125 allowing a (not shown) needle assembly to be releasably mounted to the main drug delivery device 105. Alternatively, as shown in FIG. 5A, distal coupling means 126 allowing a needle assembly to be releasably mounted may be provided at the distal end of cartridge 130.

The cartridge holder 120 comprises proximal coupling means 121 adapted to engage corresponding coupling means arranged on an interior surface of the pen cap 110. Alternatively, the pen cap 110 may be designed to engage suitable coupling means arranged on the distal end of the proximal body portion 101.

The cartridge 130 is provided with a piston 132 driven by a piston rod 106 (see FIG. 6A) forming part of the drug expelling mechanism of the proximal body portion 101.

The cartridge 130 may for example contain an insulin, GLP-1 or growth hormone formulation.

A proximal-most rotatable dose setting member 180 serves to manually set a desired dose of drug. The dose set by the user may be shown in a display window 102, The set dose can then be expelled when release button 190 is actuated.

Depending on the type of drug expelling mechanism embodied in the drug delivery device 100, the expelling mechanism may comprise a spring as in the shown embodiment, which is strained during dose setting and then released to drive the piston rod 106 when the release button 190 is actuated. Alternatively the expelling mechanism 105 may be fully manual in which case both the dose setting member 180 and the actuation button 190 move proximally during dose setting corresponding to the set dose size, and then is moved distally by the user to expel the set dose, e.g. as in a FlexPen® manufactured and sold by Novo Nordisk A/S.

Although FIGS. 1-4 show a drug delivery device/assembly 100 of the prefilled type, i.e. it is supplied with a pre-mounted cartridge 130 and is to be discarded when the cartridge 130 has been emptied, the present invention also concerns a drug delivery device/assembly 100 designed to allow cartridges 130 to be replaced, e.g. in the form of a “rear-loaded” drug delivery device/assembly 100, in which the cartridge holder 120 is adapted to be removed from the proximal body portion 101, or alternatively in the form of a “front-loaded” device in which a cartridge is inserted through a distal opening in the cartridge holder 120, which may in this case be non-removably attached to the proximal body part 101 of the device, such a device being known from e.g. WO 2014/187814.

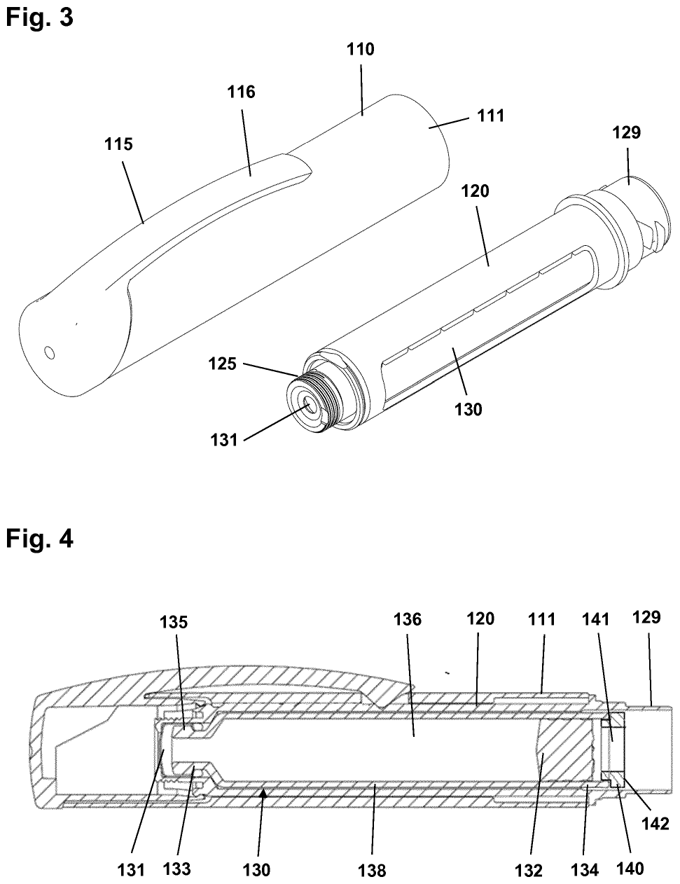

FIG. 3 shows a pen cap/cap part 110 in combination with a cartridge holder 120 in which a generally tubular drug-filled cartridge 130 with a septum outlet 131 is arranged.

The cap part 110 has a general tubular configuration with a closed distal end and an open proximal end adapted to axially receive the cartridge holder 120.

The pen cap 110 is adapted to be releasably secured to the cartridge holder 120 to cover the drug outlet. The pen cap 110 comprises a generally tubular housing portion 111 as well as a flexible clip portion 115 having a free end 116. FIG. 4 shows the pen cap 110 fully mounted and secured on the cartridge holder 120. The above-described clip portion 115 allows a pen device with the cap mounted to be attached to e.g. a shirt pocket.

The cartridge holder 120 generally corresponds to the cartridge holder 120 described in connection with FIGS. 1 and 2 described above. However, in the FIG. 3 embodiment the cartridge holder 120 comprises a proximal coupling portion 129 adapted to releasably engage the distal end of the proximal body portion 101, e.g. as described in connection with FIGS. 1 and 2 above. Separation of the cartridge holder 120 from the proximal body portion 101 allows access to the interior of the cartridge holder 120 and thereby allow exchange of the cartridge 130. Alternatively, the cartridge holder 120 may be in the form of a disposable unit comprising a non-removable drug-filled cartridge and the coupling means 129 allowing the cartridge 130 to be mounted on and released from a durable device housing portion, such as the proximal body portion 101 discussed above. The cartridge 130 may be held in place by snap means arranged to grip the cartridge 130, e.g. engaging the neck portion 133 and/or the distal-most edge portion of the cartridge 130.

The cartridge holder 120 preferably has a general tubular configuration defining an axis of reference. The cartridge holder 120 further has a central hollow configured for receiving a cartridge 130.

The cartridge 130 comprises an elongate cartridge body with a generally cylindrical main portion defining a longitudinal axis, a distal outlet portion 135 with an outlet opening, and a proximal edge portion 134 having a circumferential inner surface and a proximal opening surrounded by a circumferential edge. In the shown embodiment the cartridge axis is arranged in the central hollow of the cartridge holder 120 corresponding to the axis of reference of the cartridge holder 120. The cartridge 130 forms a central hollow 136 configured for containing a drug, such as mentioned above.

The cartridge has a tubular sidewall 138 extending between the proximal and the distal end portions 134, 135. At the distal end 135 the cartridge 130 may be formed with a neck portion 133. Further, a distal drug outlet in the form of a needle-penetrable septum 131 is formed at the distal end 135 of the cartridge 130.

The cartridge 130 further comprises a piston 132 which has a cross sectional shape and size configured to cooperate with the cross-sectional shape and size of the inner surface of the sidewall 138 of the cartridge 130 in such a way that a drug in liquid form may be contained between the piston, the sidewall 138 and the needle penetrable septum 131. When a needle assembly (not shown) has been mounted to penetrate the septum 131, the drug contained in the cartridge 130 may be expelled from the cartridge 130 by moving the piston 132 in the distal direction. The piston 132 may be moved (driven) by a piston rod 106 forming part of the drug expelling mechanism of the main drug delivery device 105, this as illustrated in FIGS. 6A and 6B.

A ring-formed code member 140 is arranged at the proximal end 134 of the cartridge 130. The code member 140 as shown in FIG. 4 has as through-going drive opening 141 formed therein and configured for receiving a piston rod 106 in driving engagement with piston 132.

FIG. 5A shows a side view of a cartridge 130 with a code member 140 according to an embodiment of the invention. FIG. 5B shows in a sectional view details of proximal end portion 134 of cartridge 130 with an attached code member 140. The ring-formed code member 140 is attached to the circumferential edge portion and comprises an axially extending mounting portion 144 in engagement with the edge portion circumferential inner surface, a flange portion 145 having a distally facing mounting surface in engagement with the circumferential cartridge edge and a proximally facing stop surface 142, as well as drive opening 141 allowing a piston rod to be received and engage the piston proximal surface. In the shown embodiment both code member portions 144, 145 have a generally ring-formed configuration, however, in alternative embodiments each or both portions may be formed by separate sections. The mounting portion has an outer surface and a cross-sectional form and size configured such that the outer surface of the connecting flange 144 cooperates with the internal surface of the sidewall of the cartridge 130 in such a manner that the code member 140 may be attached to the proximal end 134 of the cartridge 130 by a frictional fit. Alternatively or additionally the code member may be attached by adhesive means.

As shown in FIG. 4 the flange portion radially extends within the circumferential boundaries of the cartridge main portion, this allowing the cartridge to be received in an unmodified cartridge holder. In the shown embodiment the flange portion is in the form of a circular ring with an outer diameter generally corresponding to the outer diameter of the cartridge.

As described above, the code member 140 has as through-going piston rod drive opening 141 formed therein, which is configured for receiving a piston rod 106 distal drive end. As shown in FIGS. 6A and 6B the piston rod distal end may comprise a piston washer 107 attached at the distal end of the piston rod per se. Proximally the piston rod is attached to and cooperate with the drug expelling mechanism per se (not shown), e.g. by means of longitudinal drive grooves and a thread along the length of the rod.

The flange portion 145 further comprises a proximally facing circumferential surface 142 surrounding the through-going piston rod opening 141.

The piston rod drive opening 141 may by circular with a diameter D. It will be appreciated that the size of the piston rod opening 141 will only allow the passage of piston rods 106 (or piston rod washers 107) with a (circumscribed) diameter, d1, d2, which is smaller than the diameter D of the piston rod opening 141 in the code member. Thereby, the code member 140 may serve as a mechanical coding. Alternatively the opening and the washer may be configured as corresponding “key and slot” structures, e.g. having an irregular form, however, this would typically make insertion of the piston rod distal end into the cartridge more difficult.

Referring to FIGS. 6A and 6B a piston washer 107 is mounted rotatably free on the piston rod 106 distal end. The piston washer 107 shown in FIG. 6A has a diameter d1 corresponding (almost) to the diameter D of the piston rod opening 141 of the code member 140. This will allow the piston washer 107 and thereby the piston rod 106 to be inserted through the opening 141 in the code member 140, and thereby to engage and move the piston 132 in the axial direction of the cartridge 130, this allowing a drug content contained in the interior 136 of the cartridge may be expelled from the cartridge through a mounted hollow needle.

FIG. 6B shows a situation, where a cartridge 130 has a code member 140 with a piston rod opening 141 sized and shaped similar to the piston rod opening 141 in FIG. 6A, e.g. a diameter D. However, in this situation the piston washer 107 has a larger diameter d2 than diameter d1 of the piston washer shown in FIG. 5A. As diameter d2 is larger than diameter D of the piston rod opening 141 of the code member 140, the piston washer 107 and the piston rod 106 shown in FIG. 6B is prevented from being inserted into cartridge 130, and thereby from making contact with and move the piston 132. In this case a drug content contained in the cartridge 130 cannot be expelled therefrom.

Thereby, in a first embodiment, a code member 140 with opening 141 is used to code a drug cartridge 130. The code member 140 reduces the piston rod opening diameter D relative to cartridges without a code member for the piston washer 107 of the drug expelling mechanism of the main drug delivery device 105. With two different piston washers 107 having diameters d1 and d2, the larger diameter washer 107 will be blocked by the code member 140 and prevent a potential mix-up of drug types.

The shown example of a coding structure a specific size openings D in the code member and a corresponding diameter d1 of the piston rod 106 or piston washer 107 may indicate e.g. a long-acting insulin formulation whereas a coding structure having e.g. a diameter d2 of the piston rod 106 or piston washer 107 may indicate a fast-acting insulin. However, the coding structure and the individual structures can be varied in numerous ways. For example, the diameters may have any desired size.

The implementation of the current invention could prevent that a high concentration drug cartridge (or fast-acting insulin) is used in a pen device 100 reserved for normal concentration drugs (or, long-acting insulin). Hence, a major risk of mix-up incidents is mitigated.

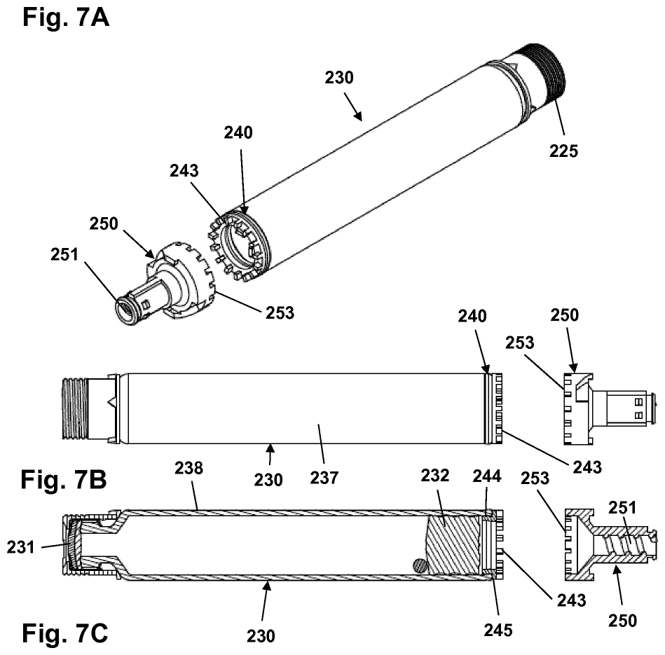

Turning to FIGS. 7A-7C a second embodiment of a drug delivery system including one or more cartridges 230 with code members 240 and one or more drug delivery devices comprising a corresponding mating member 250 adapted to engage a cartridge code member.

Cartridge 230 generally corresponds to the above-described cartridge 130 comprising an attached code member 240. The ring-formed code member 240 is attached to the circumferential edge portion and comprises an axially extending mounting portion 244 in engagement with the edge portion circumferential inner surface, a flange portion 245 having a distally facing mounting surface in engagement with the circumferential cartridge edge and a proximally facing stop surface provided with a code configuration 243 in the form of a number of circumferentially arranged axially oriented protrusions, as well as drive opening 241 allowing a piston rod to be received and engage the piston proximal surface. In the shown embodiment both code member portions 244, 245 have a generally ring-formed configuration, however, in alternative embodiments each or both portions may be formed by separate sections. The mounting portion has an outer surface and a cross-sectional form and size configured such that the outer surface of the mounting portion 244 cooperates with the internal surface of the sidewall of the cartridge 230 in such a manner that code member 240 may be attached to the proximal end of the cartridge by a frictional fit. Alternatively or additionally the code member may be attached by adhesive means.

As shown in FIGS. 7B and 7C the flange portion 245 radially extends within the circumferential boundaries of the cartridge main portion, this allowing the cartridge to be received in an unmodified cartridge holder. In the shown embodiment the flange portion is in the form of a circular ring with an outer diameter generally corresponding to the outer diameter of the cartridge.

The mating member 250 forms part of a main drug delivery device part comprising a piston rod drive mechanism. In the shown embodiment the mating member is represented by a combined nut and coupling member having an internal thread 251 adapted to engage a correspondingly threaded piston rod, the combined nut and coupling member being arranged to move axially during a cartridge exchange procedure, e.g. when a cartridge holder is removed, the axial movement controlling a coupling allowing the piston rod to be moved proximally, e.g. as disclosed in WO 2008/074897.

In the shown embodiment the mating member is provided with a circumferential array of distally extending projections forming a mating code configuration adapted to engage a cartridge stop surface code configuration 243 in mating or non-mating relationship. Alternatively, the mating code configuration could be formed on any suitable distally facing structure in the drug delivery device arranged to engage the cartridge distal end.

For a mating combination of a cartridge and a corresponding drug delivery device the cartridge code configuration would be axially received in the mating code configuration allowing the cartridge to be positioned in a mated proximal-most position, this position allowing a cartridge holder to be actuated from a receiving state to a holding state in which an inserted cartridge is held in an operational position.

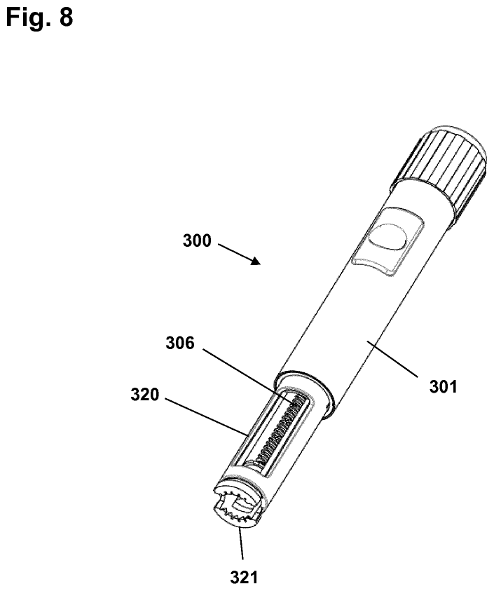

For example, for a rear-loaded cartridge holder 120 (see FIG. 3) an axially non-seated cartridge would prevent the cartridge holder to be fully mounted on the pen body. For a drug delivery device 300 comprising a front-loaded cartridge holder 320 (see FIG. 8) an axially non-seated cartridge would prevent the cartridge holder distal holding means 321 to be fully closed when actuated by e.g. rotational movement of the cartridge holder relative to the body housing 301.

In the shown embodiment the form of the code configurations 243, 253 are mainly for illustrative purposes as the angular design would make mating somewhat difficult during normal use. Correspondingly, a more user-friendly design would comprise inclined surfaces adapted to rotate the mating structures into engagement.

As appears, in contrast to the first embodiment of FIGS. 6A and 6B in which a larger diameter washer was prevented from being received in a smaller diameter cartridge, but in which a larger diameter cartridge would receive both a large and small diameter washer, the second embodiment would allow cartridges and drug delivery devices to be specifically coded allowing only a specifically coded cartridge to be received in a correspondingly coded drug delivery device.

Indeed, a given code member may be provided with both of the above-described code structures, i.e. a coded opening as well as a coded stop surface.

In the above description of exemplary embodiments, primarily situations in which a user is prevented from incorrectly mounting a given drug cartridge in a given drug delivery device is described, however, the coding concept of the present invention could also be utilized in the manufacturing of prefilled devices. For example, if cartridges are filled and manufactured in one facility and then shipped to a second facility coding of the cartridges by the addition of an inexpensive code member may help prevent a given batch of cartridges to be mounted in the wrong type of device.

In the above description of exemplary embodiments, the different structures and means providing the described functionality for the different components have been described to a degree to which the concept of the present invention will be apparent to the skilled reader.

The detailed construction and specification for the different components are considered the object of a normal design procedure performed by the skilled person along the lines set out in the present specification.

1. A cartridge, comprising:
a cartridge body comprising a generally cylindrical main portion defining a longitudinal axis, a distal outlet portion with an outlet opening, and a proximal edge portion having a circumferential inner surface and a proximal opening surrounded by a circumferential edge,
a needle penetratable septum arranged to close and seal the outlet opening,
a piston slidable received in the cylindrical main portion and comprising a proximal free surface, a variable-volume reservoir being formed between the septum and the piston member, and
a ring-formed code member attached to the circumferential edge portion and comprising:
an axially extending mounting portion in engagement with the edge portion circumferential inner surface,
a flange portion having a proximally facing stop surface, and
a drive opening allowing a piston rod to engage the piston proximal surface,
wherein the flange portion radially extends within the circumferential boundaries of the cartridge main portion.

2. A cartridge as in claim 1, wherein the flange stop surface has a non-planar configuration comprising a code pattern of axially extending code structures.

3. A cartridge as in claim 1, wherein the cartridge body is made of glass and the code member is made of a polymeric material.

4. A drug delivery system comprising:
a first cartridge and a second cartridge as in claim 1, the code member of the first cartridge having a first drive opening with a first code configuration, and the code member of the second cartridge having a second drive opening with a second code configuration,
a first drug delivery device, comprising:
a cartridge holder adapted to receive either of the first and the second cartridge,
a first piston rod having a distal end adapted to engage and axially displace a piston in a loaded cartridge in a distal direction to thereby expel a dose of drug from the cartridge, the first piston rod distal end having a first code configuration, and
a drive assembly adapted to move the first piston rod in the distal direction,
wherein the first piston rod code configuration allows the first piston rod to be inserted through the first drive opening but prevents it from being inserted through the second drive opening.

5. A drug delivery system as in claim 4, further comprising:
a second drug delivery device, comprising:
a cartridge holder adapted to receive either of the first and the second cartridge,
a second piston rod having a distal end adapted to engage and axially displace a piston in a loaded cartridge in a distal direction to thereby expel a dose of drug from the cartridge, the second piston rod distal end having a second code configuration, and
a drive assembly adapted to move the second piston rod in the distal direction, wherein:
the first piston rod code configuration allows the first piston rod to be inserted through the first drive opening but prevents it from being inserted through the second drive opening, and
the second piston rod code configuration allows the second piston rod to be inserted through the second drive opening but prevents it from being inserted through the first drive opening.

6. A drug delivery system, comprising:
a first cartridge and a second cartridge as in claim 1, the code member of the first cartridge having a first stop surface with a first code configuration, and the code member of the second cartridge having a second stop surface with a second code configuration,
a first drug delivery device, comprising:
a housing,
a cartridge holder adapted to receive and hold the cartridge, the cartridge holder being actuatable between:
(i) a receiving state in which a cartridge can be inserted and received in an axial direction, and
(ii) a holding state in which an inserted cartridge is held in an operational position,
a piston rod having a distal end adapted to engage and axially displace a piston in a loaded cartridge in a distal direction to thereby expel a dose of drug from the cartridge,
a drive assembly arranged in the housing and adapted to move the first piston rod in the distal direction, and
a first distally facing mating structure having a first mating code configuration adapted to axially engage a cartridge stop surface code configuration,
wherein the first mating code configuration allows the first stop surface to be received in the first mating structure to an axial position allowing the cartridge holder to be actuated from the receiving state to the holding state, but prevents the second stop surface to be received in the first mating structure to an axial position allowing the cartridge holder to be actuated from the receiving state to the holding state.

7. A drug delivery system as in claim 6, further comprising:
a second drug delivery device, comprising:
a housing,
a cartridge holder adapted to receive either of the first and second cartridge, the cartridge holder being actuatable between:
(i) a receiving state in which a cartridge can be inserted and received in an axial direction, and
(ii) a holding state in which an inserted cartridge is held in an operational position,
a piston (x) rod having a distal end adapted to engage and axially displace a piston in a loaded cartridge in a distal direction to thereby expel a dose of drug from the cartridge,
a drive assembly arranged in the housing and adapted to move the first piston rod in the distal direction, and
a second distally facing mating structure having a second mating code configuration adapted to axially engage a cartridge stop surface code configuration,
wherein:
the first mating code configuration allows the first stop surface to be received in the first mating structure to an axial position allowing the cartridge holder to be actuated from the receiving state to the holding state, but prevents the second stop surface to be received in the first mating structure to an axial position allowing the cartridge holder to be actuated from the receiving state to the holding state, and
the second mating code configuration allows the second stop surface to be received in the second mating structure to an axial position allowing the cartridge holder to be actuated from the receiving state to the holding state, but prevents the first stop surface to be received in the second mating structure to an axial position allowing the cartridge holder to be actuated from the receiving state to the holding state.

8. A drug delivery system as in claim 6, wherein for each drug delivery device the cartridge holder and the housing comprise corresponding coupling structure allowing the cartridge holder to be removed from the housing corresponding to the receiving state and mounted on the housing corresponding to the holding state.

9. A drug delivery system as in claim 8, wherein the coupling structure is in the form of a bayonet coupling or a threaded coupling.

10. A drug delivery system as in claim 6, wherein for each drug delivery device the cartridge holder is non-removable from the housing, the cartridge holder comprising a distal opening allowing a cartridge to be received in a proximal direction, the cartridge holder comprises holding structure actuatable from:
(i) a receiving state allowing a cartridge to be inserted into the cartridge holder, to
(ii) a holding state in which a received cartridge is held in an operational position.

11. A drug delivery system as in claim 10, wherein the holding structure comprises one or more locking members each having a distal gripping portion being moved proximally when the holding structure is actuated from the receiving state to the holding state.
